(19) United States
(12) Patent Application Publication
(10) Pub. No.: US 2020/0000632 A1
(43) Pub. Date: Jan. 2, 2020

(54) Simultaneous Thermal and Cooling Therapeutic Device

(71) Applicants: Bruce Wayne Miller, Naples, FL (US); Wayne Allan Miller, Land O lakes, FL (US)

(72) Inventors: Bruce Wayne Miller, Naples, FL (US); Wayne Allan Miller, Land O lakes, FL (US)

(21) Appl. No.: 16/244,498

(22) Filed: Jan. 10, 2019

Related U.S. Application Data
(60) Provisional application No. 62/691,455, filed on Jun. 28, 2018.

(51) Int. Cl. A61F 7/02; A61F 7/10

(52) U.S. Cl. CPC A61F 7/02; A61F 2007/0087; A61F 7/10

(57) ABSTRACT

A therapeutic device combines both hot and cold therapy to work simultaneously together. The therapeutic device has a first portion being made of a material capable of being heated to a first temperature and a second portion having a fastener capable of connecting to a cold pack having a second temperature. The application of both hot and cold to the site of an injury of a user stimulates blood circulation to an injured muscle with heat and reduces inflammation with cold to facilitate a faster and more efficient recovery process of the afflicted area.

### CROSS REFERENCE TO RELATED APPLICATIONS

This application is an original Non-Provisional Utility application entitled, “Simultaneous Thermal and Cooling Therapeutic Device”, which claims priority to co-pending U.S. Provisional Patent Application No. 62/691,455, filed Jun. 28, 2018, entitled, “HC Therapy”.

### BACKGROUND OF THE INVENTION

### 1. Field of the Invention

This invention relates, generally, to a simultaneous thermal and cooling therapeutic device. More particularly, it relates to a thermal apparatus having a removable cold pack.

### 2. Background Art

This novel therapeutic device is configured to help injuries associated with including, but not limited to, sore muscles, sprained muscles, or pulled muscles. The therapeutic device when applied to the injury reduces inflammation with ice or cold packs while also stimulating circulation in the area with hot packs, simultaneously. Many therapeutic devices in today's market either use hot or cold to help with symptoms associated with injury. However, these therapeutic devices do not use bot hot and cold at the same time. Using hot and cold together can result in a faster and more efficient way to reduce inflammation, therefore reducing the amount of therapy or product needed to alleviate the pain of the area. Thus, there is a need for a therapeutic device that combines the use of hot and cold in a single therapeutic device in an attempt to save time and energy, to provide more comfort to a user, and to more effectively reduce injury mediated inflammation.

Currently, when an individual has a muscle injury, they use either ice alone or heat alone to treat it. In an example of a muscle injury treatment method, contrast therapy is the application of heat for approximately 20 minutes followed by an application of cold such as a cold pack or ice for approximately 20 minutes. There is a need for a novel therapeutic device configured to combine a cold and hot pack together to deliver simultaneous hot and cold therapy to a user. As a result, the novel therapeutic device is configured to reduce inflammation and stimulate more blood flow to the injured or swollen area. The sensation a user experiences from using a combined hot and cold therapeutic device relaxes the nerves associated with the brains response to injury. For example, by using hot and cold at the same time, the signals of the brain become confused and may alleviate the area of injury quicker.

In another example of a muscle injury treatment method, cold therapy is effective for pain and inflammation reduction. Cold cools the skin tissue which results in narrowing of blood vessels in a process vasoconstriction. Vasoconstriction reduces swelling, reduces inflammation, and reduces muscle spasms of the injured area. There is a need to take a standard cold pack and combine it together with a hot pack, resulting in a contrast therapy that works at the same time.

In another example of a muscle injury treatment method, heat therapy effectively increases tissue extensibility and improves muscle movement after an injury. Heat therapy also induces vasodilation which draws blood into targeted tissues and increases blood flow. This increase in blood flow is beneficial because the blood flow delivers required oxygen and nutrients to the injured area as well as removes cellular waste. The warmth of heat therapy is associated with a decreases muscle spasms, relaxes tense muscles, relieves pain, and may increase the range in motion. Heat therapy also stimulates the sensory receptors in the skin, resulting in a decrease in transmission of pain signals to the brain in an attempt to relieve discomfort during an injury. Thus, heat therapy promotes healing of the muscles and joints. There is a need to take a standard hot pack and combine it together with a cold pack, resulting in a contrast therapy that works at the same time.

However, in view of the prior art considered as a whole at the time the present invention was made, it was not obvious to those of ordinary skill in the pertinent art how the identified needs could be fulfilled.

### SUMMARY OF THE INVENTION

The long-standing but heretofore unfulfilled need for a therapeutic device configured to combine both hot and cold therapy to work simultaneously together. The therapeutic device has a first portion being made of a material capable of being heated to a first temperature and a second portion having a fastener capable of connecting to a cold pack having a second temperature. The application of both hot and cold to the site of an injury of a user stimulates blood circulation to an injured muscle with heat and reduces inflammation with cold to facilitate a faster and more efficient recovery process of the afflicted area. and is now met by a new, useful, and non-obvious invention.

The following description and drawings are illustrative and are not to be construed as limiting. Numerous specific details are described to provide a thorough understanding. However, in certain instances, well know or conventional details are not described in order to avoid obscuring the description. References to one or an embodiment in the present disclosure are not necessarily references to the same embodiment; and, such references refer to at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

In an alternate embodiment, the standard hot packs and cold packs can be combined together with a fastener, including, but not limited to, hook and loop attachments, a slit of fabric, a plastic pocket, or a magnet.

In a preferred embodiment, a therapeutic device is shaped like a large donut sized pack. The outer part of the pack will be for heat that is configured to be heated by a user in a microwave. The inner part will have at least a portion of a piece of hook and loop attachments sewn, a slit of fabric or a magnet that is configured for a user to place a cold pack into the center and stay secured. This therapeutic device can then be placed on the injury and both the heat and cold will work together to stimulate blood flow and reduce inflammation to the afflicted area.

It is within the scope of this invention for the novel therapeutic device to have any shape and size including, but not limited to, a donut shape, rounded, oval, square, or rectangular. The novel therapeutic device operates with one portion being hot and another portion having a fastener, including, but not limited to, hook and loop attachments, a slit of fabric, a plastic pocket, or a magnet configured to receive a cold pack.

In an alternate embodiment, the novel therapeutic device is a shape including, but not limited to, a square or a rectangular shape having a plurality of pockets connected thereto. For example, the therapeutic device can have four different slots each positioned in a horizontal orientation. Each of the four different slots are configured to receive four hot and cold packs alternately positioned.

The novel simultaneous thermal and cooling therapeutic device is a donut shape having an outer perimeter edge. The outer perimeter edge is a first pack capable of being heated to a first temperature. The therapeutic device can be heated in a microwave, which would warm the outer perimeter edge portion to a warm or hot first temperature. The therapeutic device has a substantially central portion that does not have a portion of a first pack and does not have a heated temperature. The substantially central portion of the therapeutic device is configured to receive a removable second pack capable of being cooled to a second temperature. The first temperature of the first pack is of a different temperature than the second temperature of said second pack.

It is within the scope of this invention for the therapeutic device to have an outer perimeter edge being a first pack capable of being cooled to first temperature. Additionally, it is also within the scope of this invention for the substantially central portion of the therapeutic device to be configured to receive a removable second pack capable of being heated to a second temperature.

In another embodiment, the simultaneous thermal and cooling therapeutic device has an outer perimeter edge being a first pack made of a thermally conductive material or being made of any material capable of retaining a thermally conductive material. The thermally conductive material includes, but is not limited to, microwavable beads, rice, rice beads, or any material that can be safely heated up in a microwave. In an example, the outer perimeter edge of the therapeutic device is a fabric material having a pocket retaining rice. In another example, the outer perimeter edge of the therapeutic device is a plastic material having a pocket retaining a gel that is capable of being heated. In yet another example, the outer perimeter edge of the therapeutic device is a first pack being a vessel retaining water. In yet another example, the outer perimeter edge of the therapeutic device is a first pack being a vessel retaining a thermally conductive gel. In yet another example, the outer perimeter edge of the therapeutic device is a first pack being a vessel retaining a liquid. In yet another example, the outer perimeter edge of the therapeutic device is a first pack being a hot pack.

In another embodiment, the simultaneous thermal and cooling therapeutic device has a first donut shaped pack configured to be heated to a first temperature.

In another embodiment, the simultaneous thermal and cooling therapeutic device has a middle portion of the donut shaped hot pack capable of receiving a removable second pack. The removable second pack is configured to be cooled to a second temperature. It is within the scope of this invention for the removable second pack to be a vessel retaining including, but not limited to, a refrigerant gel, ice, freezable gel, water, or beads. The vessel is a container, a pouch, a pack capable of retaining the material to be cooled to the second cooler temperature. It is also within the scope of this invention for the cold pack to be made from a flexible soft plastic material on the outside that retains the substance or material configured to be cooled to a second temperature.

The material that would hold the packs can be a cotton material that is cut, shaped, and filled with the beads, then sewn in to fit the shape or pattern. The hot pack and cold pack will be separate components of the therapeutic device. After the therapeutic device having the hot pack is heated and the cold pack is cooled, the cold pack can be removably connected to the central portion of the therapeutic device. The therapeutic device can then be applied to the injured area and provide simultaneous hot and cold therapy to a user.

In another embodiment, the simultaneous thermal and cooling therapeutic device has a substantially central portion having a pocket. The pocket is configured to retain the removable second pack.

In another embodiment, the simultaneous thermal and cooling therapeutic device has a substantially central portion having a first portion of a fastener. The removable second pack has a second portion of a fastener. The first portion of the fastener removably connects to the second portion of said fastener. In an example, the first portion of a fastener is the hook portion and the second portion of a fastener is the loop portion. The hook portion mates with the loop portion to form an attachment. It is within the scope of this invention for the fastener to be an adhesive, a button, a clip, or a magnet. This allows for the cold pack to connect to the central portion of the therapeutic device, thereby, orienting the hot pack to be on the outer perimeter edge of the therapeutic device and the cold pack located in the center of the therapeutic device.

In another embodiment, the simultaneous thermal and cooling therapeutic device has a substantially central portion having a length of material. The length of material has a first end located opposite a second end. The first end of the length of material and said second end of the length of material are connected to the therapeutic device. The length of material can be an elastic material capable of stretching to retain a plurality of sexed cold packs. The length of material is configured to retain a removable second pack when the removable second pack is inserted under the length of material.

In yet another embodiment, the simultaneous thermal and cooling therapeutic device has an outer perimeter edge being a hot pack. The hot pack is made of a thermally conductive material capable of being heated to a first temperature. The therapeutic device has a substantially central portion configured to receive a removable cold pack. The removable cold pack is made of a material capable of being cooled to a second temperature. The second temperature of the removable cold pack is a lower temperature than the first temperature of the hot pack. The removable cold pack is connected to the substantially central portion of the therapeutic device.

The substantially central portion of the therapeutic device can have a pocket configured to retain the removable cold pack. In another embodiment, the substantially central portion of the therapeutic device can have a first portion of a fastener. The removable cold pack has a second portion of a fastener. The first portion of the fastener removably connects to the second portion of the fastener. It is within the scope of this invention for the second portion of the fastener to be the outer surface of the cold pack. In another embodiment, the substantially central portion of the therapeutic device has a length of material having a first end located opposite a second end. The first end of the length of material and the second end of the length of material are connected to the central portion of the therapeutic device. The length of material is configured to retain the removable cold pack when the removable cold pack is inserted under the length of material.

It is within the scope of this current invention for the removable cold pack to be reusable or an instant cold pack.

### BRIEF DESCRIPTION OF THE DRAWINGS

For a fuller understanding of the nature and objects of the invention, reference should be made to the following detailed description, taken in connection with the accompanying drawings, in which:

FIG. 1 is a side cut-away view of the novel therapeutic device having an outer perimeter edge having a compartment retaining a thermally conductive material and a substantially central portion having a first portion of a fastener receiving a removable cold pack having a second portion of a fastener connected thereto;

FIG. 2 is a top perspective view of the novel therapeutic device having an outer perimeter edge being a hot pack and a substantially central portion being a removable cold pack;

FIG. 3 is an exploded view of the novel therapeutic device having an outer perimeter edge being a hot pack and a substantially central portion being a removable cold pack;

FIG. 4 is a side cut-away view of an alternate embodiment of the novel therapeutic device having an outer perimeter edge having a compartment retaining a thermally conductive material and a substantially central portion forming a pocket with a length of material receiving a removable cold pack;

FIG. 5 is a side cut-away view of an alternate embodiment of the novel therapeutic device having an outer perimeter edge having a compartment retaining a thermally conductive material and a substantially central portion forming a pocket with a length of material being expanded to receive a removable cold pack;

FIG. 6 is a side cut-away view of another alternate embodiment of the novel therapeutic device having an outer perimeter edge having a compartment retaining a thermally conductive material and a substantially central portion forming a pocket with a length of material receiving a removable cold pack;

FIG. 7 is a top view of another alternate embodiment of the novel therapeutic device having an outer perimeter edge having a compartment retaining a thermally conductive material and a substantially central portion forming a pocket with a length of material receiving a removable cold pack;

FIG. 8 is a top perspective view of an alternate embodiment of the novel therapeutic device having a square shape and having an outer perimeter edge being a hot pack and a substantially central portion being a removable cold pack; and,

FIG. 9 is a side perspective view of an alternate embodiment of the novel therapeutic device having a rectangular shape with a plurality of pockets configured to receive alternating removable hot packs and removable cold packs evenly through the therapeutic device.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

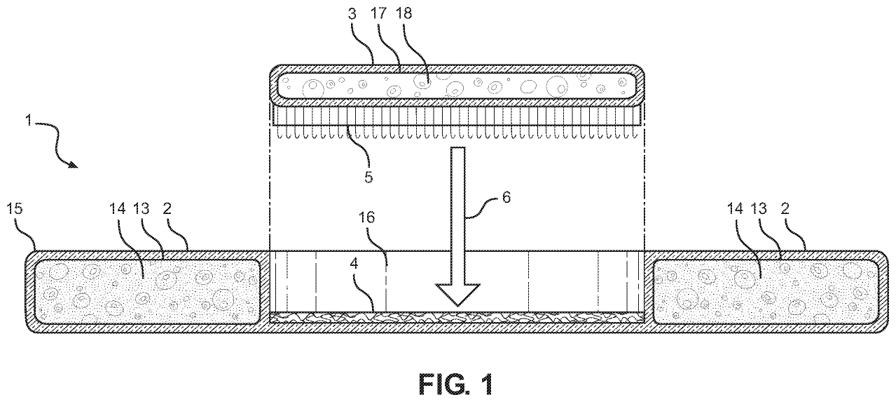

FIG. 1 illustrates novel therapeutic device 1 having hot pack 2 located on an outer perimeter edge 15 of therapeutic device 1. Hot pack 2 has compartment 13 retaining thermally conductive material 14. Therapeutic device 1 has central portion 16 forming a chamber or a recess capable of receiving 6 (FIGS. 1 and 3) removable cold pack 3. Removable cold pack 3 has compartment 17 capable of retaining refrigerant gel 18. Central portion 16 of therapeutic device 1 has first portion of fastener 4 connected to at least one inner wall surface or bottom surface of central portion 16. The first portion of fastener 4 featured in FIG. 1 are the loops of a hook and loop fastening mechanism. Removable cold pack 3 has at least a portion of a fastener 5 connected thereto. In particular, second portion of fastener 5 is the hook portion of a hook and loop fastener mechanism. Second portion of fastener 5 of removable cold pack 3 is configured to connect to 6 first portion of fastener 4 of central portion 16 of therapeutic device 1.

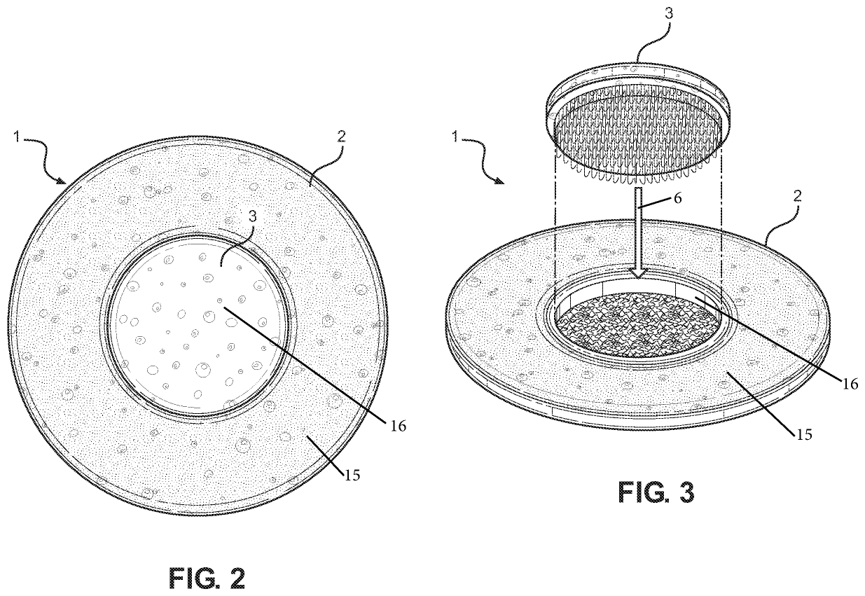

FIGS. 2-3 illustrate therapeutic deice 1 having hot pack 2 located on outer perimeter edge 15 of therapeutic device 1 and removable cold pack 3 located on central portion 16 of therapeutic device 1. FIGS. 2-3 illustrate different views of FIG. 1. Although not shown, it is within the scope of this invention for the cold pack to be located on an outer perimeter edge of the therapeutic device and a removable hot pack located on a central portion of the therapeutic device.

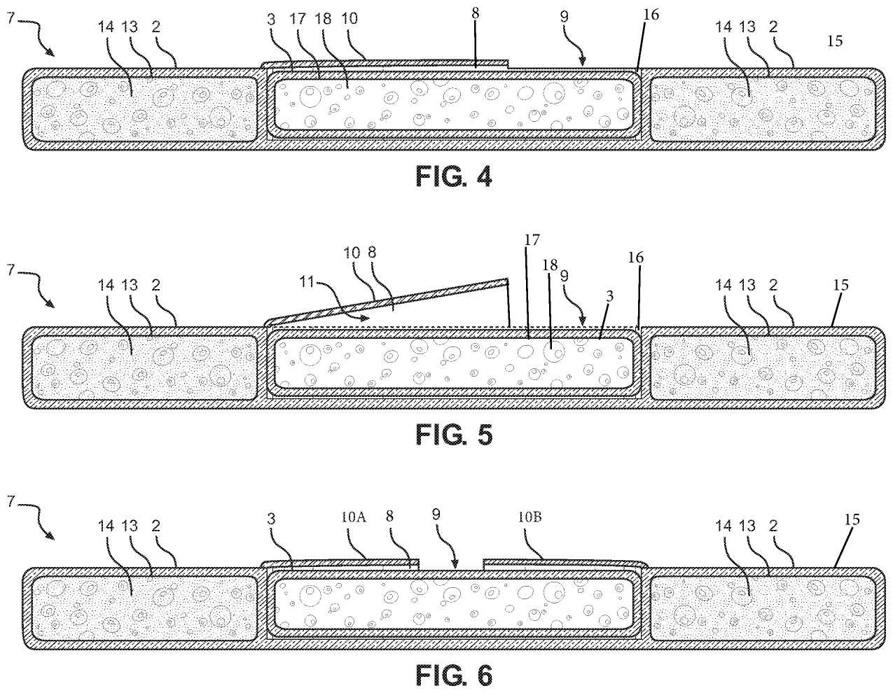

FIGS. 4-5 depict an alternate embodiment of therapeutic device 7 in which the fastener connecting the removable cold pack 3 to therapeutic device 1 is a length of material 10 partially overlaying at least a portion of pocket 8. Pocket 8 is located at central portion 16 of therapeutic device 7. Pocket 8 has pocket opening 9. In this embodiment, opening 9 is located on an edge of central portion 16. It is within the scope of this invention for removable cold pack 3 having a compartment 17 retaining refrigerant gel 18. Therapeutic device 7 has hot pack 2 located on an outer perimeter edge 15 of therapeutic device 7. Hot pack 2 has compartment 13 retaining thermally conductive material 14. FIG. 4 illustrates length of material 10 in a closed orientation being made of any material including, but not limited to, a stretchy material capable of retaining removable cold pack 3 within pocket 8. FIG. 5 illustrates length of material 10 being oriented in an open configuration 11 to allow for more clearance for removable cold pack 3 to slide into pocket 8. A user may grasp length of material 10 and peel it or pull it to create a larger opening 9 of pocket 8. For example, a user (not shown) may grasp at least a portion of length of material 10 and pull the length of material 10 away from central portion 16 to create a larger opening 9 for removable cold pack 3 to be inserted into pocket 8.

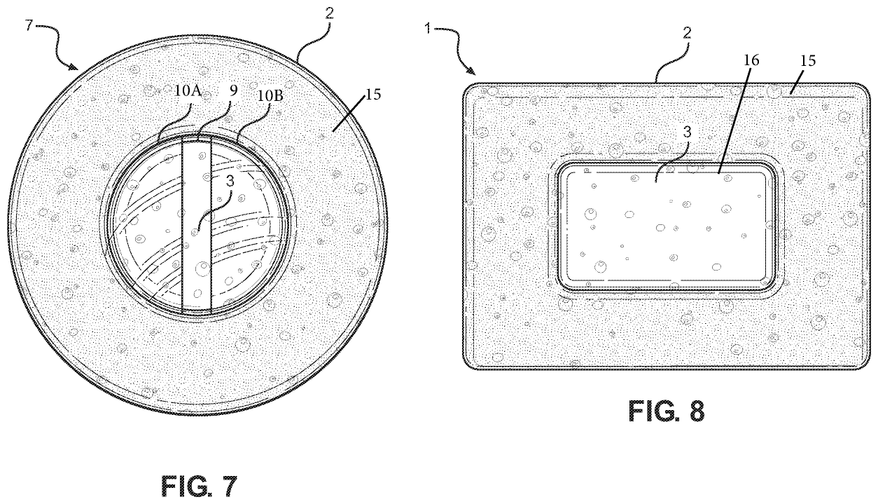

FIGS. 6-7 illustrate an alternate embodiment of therapeutic device 7 in which the fastener holding removable cold pack 3 within pocket 8 (FIG. 6) is a length of material 10A and 10B overlaying at least a portion of pocket 8 (FIG. 6). Removable cold pack 3 is inserted into pocket 8 through opening 9. In this embodiment, opening 9 is located at a central portion of central portion 16 between length of material 10A and 10B. Therapeutic device 7 has outer perimeter edge 15 being hot pack 2. FIG. 6 shows hot pack 2 having compartment 13 retaining thermally conductive material 14.

FIG. 8 shows an alternate embodiment of the novel therapeutic device 1 in FIGS. 1-3 having a square shape and having an outer perimeter edge 15 being hot pack 2 and substantially central portion 16 being removable cold pack 3.

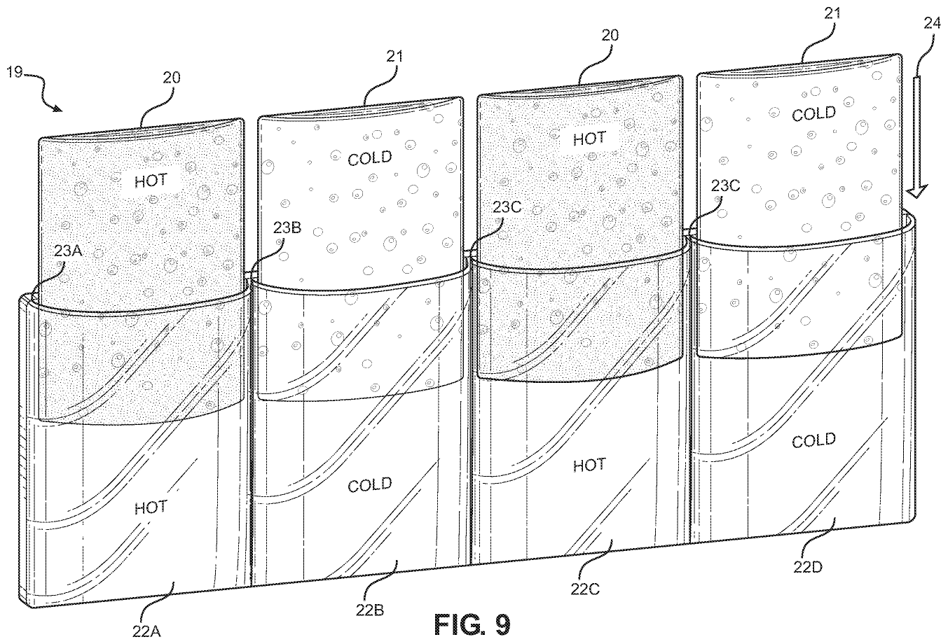

FIG. 9 is an alternate embodiment of novel therapeutic device 19 having a rectangular shape with a plurality of pockets 22A-22D each having pocket openings 23A-23D respectively. Pocket openings 23A-23D are configured to receive alternating removable hot packs 20 and removable cold packs 21 evenly through therapeutic device 19. For example, removable hot packs 20 and removable cold packs 21 slide into 24 pocket openings 23A-23D.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

The invention claimed is:

1. A simultaneous thermal and cooling therapeutic device, comprising:
said therapeutic device having an outer perimeter edge, said outer perimeter edge is a first pack having a first temperature;
said therapeutic device having a substantially central portion configured to receive a removable second pack having a second temperature; and,
said first temperature of said first pack is of a different temperature than said second temperature of said second pack.

2. The simultaneous thermal and cooling therapeutic device of claim 1, further comprising said outer perimeter edge of said therapeutic device being made of a thermally conductive material.

3. The simultaneous thermal and cooling therapeutic device of claim 1, further comprising said first pack configured to be heated to said first temperature.

4. The simultaneous thermal and cooling therapeutic device of claim 3, further comprising said first pack having a compartment retaining water.

5. The simultaneous thermal and cooling therapeutic device of claim 3, further comprising said first pack having a compartment retaining a thermally conductive gel.

6. The simultaneous thermal and cooling therapeutic device of claim 3, further comprising said first pack having a compartment retaining a liquid.

7. The simultaneous thermal and cooling therapeutic device of claim 3, further comprising said first pack is a hot pack.

8. The simultaneous thermal and cooling therapeutic device of claim 1, further comprising said removable second pack is configured to be cooled to said second temperature.

9. The simultaneous thermal and cooling therapeutic device of claim 8, further comprising said removable second pack having a compartment retaining a refrigerant gel.

10. The simultaneous thermal and cooling therapeutic device of claim 8, further comprising said removable second pack having a compartment retaining ice.

11. The simultaneous thermal and cooling therapeutic device of claim 8, further comprising said removable second pack having a compartment retaining water.

12. The simultaneous thermal and cooling therapeutic device of claim 1, further comprising said substantially central portion of said therapeutic device having a pocket, said pocket configured to retain said removable second pack.

13. The simultaneous thermal and cooling therapeutic device of claim 1, further comprising said substantially central portion of said therapeutic device having a first portion of a fastener, said removable second pack having a second portion of a fastener, said first portion of said fastener removably connects to said second portion of said fastener.

14. The simultaneous thermal and cooling therapeutic device of claim 1, further comprising said substantially central portion of said therapeutic device having a length of material having a first end located opposite a second end, said first end of said length of material and said second end of said length of material are connected to said therapeutic device, said length of material is configured to retain said removable second pack when said removable second pack is inserted under said length of material.

15. A simultaneous thermal and cooling therapeutic device, comprising:
said therapeutic device having an outer perimeter edge is a hot pack, said hot pack being made of a thermally conductive material capable of being heated to a first temperature;
said therapeutic device having a substantially central portion configured to receive a removable cold pack, said removable cold pack is made of a material capable of being cooled to a second temperature, said second temperature of said removable cold pack is a lower temperature than said first temperature of said hot pack;
said removable cold pack is connected to said substantially central portion of said therapeutic device.

16. The simultaneous thermal and cooling therapeutic device of claim 15, further comprising said substantially central portion of said therapeutic device having a pocket, said pocket configured to retain said removable cold pack.

17. The simultaneous thermal and cooling therapeutic device of claim 15, further comprising said substantially central portion of said therapeutic device having a first portion of a fastener, said removable cold pack having a second portion of a fastener, said first portion of said fastener removably connects to said second portion of said fastener.

18. The simultaneous thermal and cooling therapeutic device of claim 15, further comprising said substantially central portion of said therapeutic device having a length of material having a first end located opposite a second end, said first end of said length of material and said second end of said length of material are connected to said therapeutic device, said length of material is configured to retain said removable cold pack when said removable cold pack is inserted under said length of material.

19. The simultaneous thermal and cooling therapeutic device of claim 15, further comprising said removable cold pack is reusable.

20. The simultaneous thermal and cooling therapeutic device of claim 15, further comprising said removable cold pack is an instant cold pack.
